SEQUENCE LISTING (4 sequences)

SEQ ID NO: 1 - protein, 12 aa, Homo sapiens
LIDNNKTEKEIL

SEQ ID NO: 2 - protein, 15 aa, Homo sapiens
TKLIDNNKTEKEILD

SEQ ID NO: 3 - protein, 12 aa, Homo sapiens
  MOD_RES (3): any amino acid
LIXNNXTXXXXX

SEQ ID NO: 4 - protein, 14 aa, Homo sapiens
  MOD_RES (2): D-ala
TXLIDNNATEEILY

(19) United States
(12) Patent Application Publication
(10) Pub. No.: US 2010/0260734 A1
(43) Pub. Date: Oct. 14, 2010

(54) METHOD OF STIMULATING PROSAPOSIN RECEPTOR ACTIVITY

(75) Inventors: John S. O'BRIEN, La Jolla, CA (US); D. Elliot PARKS, San Diego, CA (US)

Correspondence Address: KENYON & KENYON LLP, ONE BROADWAY, NEW YORK, NY, 10004, US

(73) Assignee: MYELOS CORPORATION, East Brunswick, NJ (US)

(21) Appl. No.: 12/761,222

(22) Filed: Apr. 15, 2010

Related U.S. Application Data
(63) Continuation of application No. 11/508,033, filed on Aug. 21, 2006, now abandoned.
Application No. 11/508,033 is a continuation of application No. 10/293,819, filed on Nov. 13, 2002, now abandoned.
Application No. 10/293,819 is a continuation of application No. 09/802,617, filed on Mar. 9, 2001, now abandoned.

(51) Int. Cl. A61K 35/30

(52) U.S. Cl. 424/93.71

(57) ABSTRACT

A method for stimulating prosaposin receptor activity in a cell by transfecting the cell with a DNA or RNA molecule encoding prosaposin or a prosaposin receptor agonist. The DNA or RNA molecule is administered either in vivo or used to transfect neural cells or neural stem cells ex vivo followed by reintroduction of the cells into an individual.

### RELATED APPLICATIONS

This application is a continuation of prior application PCT/US99/20829 filed Sep. 9, 1999 which claims priority to U.S. application Ser. No. 09/149,977 filed Sep. 9, 1998 (now abandoned). The entire disclosure of the prior applications are hereby expressly incorporated by reference in their entireties.

### FIELD OF THE INVENTION

The present invention relates to a method of stimulating prosaposin receptor activity by transfecting cells with DNA or RNA encoding prosaposin or a prosaposin receptor agonist.

### BACKGROUND OF THE INVENTION

Prosaposin, a 70 kilodalton glycoprotein, is the precursor of a group of four heat-stable glycoproteins which are required for hydrolysis of glycosphingolipids by lysosomal hydrolases (Kishimoto et al., J. Lipid Res., 33:1255-1267, 1992). Prosaposin is proteolytically processed in lysosomes to generate saposins A, B, C, and D which exist as four tandem domains in prosaposin (O'Brien et al., FASEB J., 5:301-308, 1991). All four saposins are structurally similar to each other, including the placement of six cysteines, a glycosylation site and conserved proline residues.

As described in U.S. Pat. No. 5,571,787 and International Application No. PCT/US94/08453, prosaposin, saposin C and various peptides derived from or related to saposin C (18-mer and 22-mer peptides) induce neurite outgrowth, prevent neural cell death and stimulate myelination. These proteins and peptides, which are members of the group “prosaposin receptor agonists”, also promote neuroprotection and can be used to treat various neuropathies including diabetic neuropathy and taxol-induced neuropathy. The neurotrophic and myelinotrophic activity have been further localized to a 12-mer region (amino acids 18-29) of saposin C (LIDNNKTEKEIL; SEQ ID NO: 1). Immunohistochemical studies showed that prosaposin is localized to populations of large neurons including upper and lower motor neurons. Prosaposin binds to a cell surface receptor and stimulates incorporation of ³²P into several proteins.

The use of neurotrophic peptides as therapeutic agents has inherent limitations including susceptibility to proteolysis. In the nervous system, it is desirable for therapeutic agents to cross the blood brain barrier. Although the 18-mer referred to above can cross the blood brain barrier, the enhanced production of prosaposin, saposin C or a peptide related thereto by cells of the peripheral and/or central nervous system would be beneficial in the prevention and treatment of neurodegenerative and myelination disorders.

The prosaposin receptor is described in U.S. Pat. No. 5,571,787. This receptor also binds saposin C and prosaposin receptor agonists including the 12-mer referred to hereinabove. Methods of identifying prosaposin receptor agonists are described in U.S. application Ser. No. 08/896,181. Since the discovery of the neurotrophic, neuroprotective, and myelinotrophic activities of prosaposin receptor agonists, various researchers have demonstrated the in vitro and in vivo utility of various such agonists (O'Brien et al., Proc. Natl. Acad. Sci. U.S.A. 91:9593-9596, 1994; O'Brien et al., FASEB J. 9:681-685, 1994; Sano et al., Biochem. Biophys. Res. Commun. 204:994-1000, 1994; Kotani et al., J. Neurochem. 66:2197-2200, 1996; Kotani et al., J. Neurochem. 66:2019-2025; Qi et al., J. Biol. Chem. 271:6874-6880, 1996).

The therapeutic treatment of diseases using gene therapy involves the transfer and transient or stable insertion of new genetic information into cells (see Crystal et al., Science, 270:404-410, 1995 for review). The correction of a genetic defect by re-introduction of the normal allele of a gene encoding the desired function has been achieved (Rosenberg et al., New Engl. J. Med. , 323:570, 1990; Boris-Lawrie et al., Ann. N.Y. Acad. Sci., 716:59, 1994; Wivel et al., Science, 262:533, 1993).

In order to be therapeutically effective in the treatment of neurodegenerative of myelination disorders, prosaposin and prosaposin receptor agonists need to be delivered to neural cells transiently or stably. The present invention provides such delivery methods.

### SUMMARY OF THE INVENTION

One embodiment of the present invention is the use of an isolated DNA or RNA molecule operably encoding prosaposin or a prosaposin receptor agonist for treatment of neurodegenerative or myelination disorders. Preferably, the prosaposin receptor agonist is selected from the group consisting of saposin C, a peptide including amino acids 18-29 of saposin C and a peptide including the amino acid sequence shown in SEQ ID NO: 3. In one aspect of this preferred embodiment, the DNA or RNA molecule is in an expression vector. Preferably, the expression vector is selected from the group consisting of an adenoviral vector, retroviral vector, plasmid vector and plasmid-liposome vector. Advantageously, the disorder is selected from the group consisting of multiple sclerosis, spinal cord injury, macular degeneration, amyotrophic lateral sclerosis, spinal muscular atrophy, post-polio syndrome, muscular dystrophies, peripheral neuropathies, stroke and peripheral nerve injuries. In another aspect of this preferred embodiment, the disorder arises from a disorder arising from proinflammatory cytokine-induced apoptosis. Preferably, the disorder is a cerebral infarct or myocardial infarct. In another aspect of this preferred embodiment, the medicament is in a form suitable for an administration route selected from the group consisting of intravenous, intracerebrospinal, intramuscular, intradermal, subcutaneous, intracranial, epidural, topical, intranasal, transmucosal and oral. Preferably, the medicament is for a human. In another aspect of this preferred embodiment, the DNA or RNA molecule has been transfected or infected into neural cells from a mammal. Advantageously, the DNA or RNA molecule is in an expression vector. Preferably, the expression vector is selected from the group selected from the group consisting of an adenoviral vector, retroviral vector, plasmid vector and plasmid-liposome vector. In another aspect of this preferred embodiment, the cells are encapsulated. Preferably, the encapsulated cells are suitable for intrathecal or intracranial implantation. In another aspect of this preferred embodiment, the cells are neural stem cells. Preferably, the stem cells are precursors of cells selected from the group consisting of neurons, astrocytes and oligodendrocytes. Preferably, the medicament comprises a DNA molecule operably encoding the prosaposin receptor agonist.

The present invention also provides a viral vector, comprising a DNA or RNA molecule operably encoding a prosaposin receptor agonist.

Another embodiment of the invention is a method for producing recombinant prosaposin or a prosaposin receptor agonist, comprising the step of: administering to a mammal an isolated DNA or RNA molecule operably encoding prosaposin or a prosaposin receptor agonist; isolating body fluid from a mammal; and isolating the prosaposin or prosaposin receptor agonist from the body fluid. Preferably, the body fluid is selected from the group consisting of blood, milk, cerebrospinal fluid and semen.

### DETAILED DESCRIPTION OF THE PREFERRED EMBODIMENTS

The present invention includes a method for promoting neuroprotection and treating neurodegenerative or myelination disorders comprising delivering DNA or RNA molecules encoding prosaposin, prosaposin receptor agonists such as saposin C and peptides including amino acids 8-29 of saposin C, or the prosaposin receptor to neural cells either in vivo or ex vivo. In the present method, either the level of prosaposin/prosaposin receptor agonist is increased, or the level of prosaposin receptor is increased which, in turn, binds more circulating prosaposin/receptor agonist and results in an enhanced neuroprotective and/or neuritogenic effect. A receptor agonist is defined as a compound which has affinity for and stimulates physiologic activity at cell receptors normally stimulated by endogenous substances. Thus, receptor agonists both bind to the receptor and stimulate its activity. In another preferred embodiment, a DNA or RNA molecule encoding the prosaposin receptor is delivered to neural cells

A native 15-mer (TKLIDNNKTEKEILD; SEQ ID NO: 2) contained within human saposin C and including the active neurite-promoting region shown in SEQ ID NO: 1 was modified as follows to decrease its susceptibility to proteolysis in vivo: Lys 2 was replaced with D-ala to increase resistance to exopeptidases; lys 8 was replaced with ala to increase resistance to trypsin digestion; and lys 11 was deleted to increase resistance to trypsin digestion. In addition, asp 15 was replaced with tyr to provide an iodination site. Thus, the resulting peptide, TX14(A), contained no cleavage sites for trypsin or chymotrypsin.

SEQ ID NO: 1 may be modified as follows and still retain neurotrophic and myelinotrophic activity: Leu1 and Ile 2 are essential; Asp3 is any amino acid; Asn4 and Asn5 are essential; Lys6 is any amino acid, preferably not lysine or arginine; Thr7 is essential; Glu8 is a charged amino acid; Lys 9 is absent or a charged amino acid; Glu10 is any charged amino acid; Ile11 and Leu 12 are any amino acid. These guidelines produce the following consensus sequence:

LIX₁NNX₂TX₃X₄X₅X₆X₇ (SEQ ID NO: 3)

DNA or RNA molecules encoding prosaposin or a prosaposin receptor agonist are used to transfect or infect neural cell populations, either transiently or stably, where they continuously produce the prosaposin or prosaposin receptor agonist if under the control of a constitutive promoter, or transiently produce the prosaposin or prosaposin receptor agonist if under the control of an inducible promoter. The enhanced intracellular production of prosaposin or prosaposin receptor agonists increases prosaposin activity levels by stimulating the prosaposin receptor and initiating a cascade of events leading to, among other things, neuroprotection, inhibition of neural degeneration and inhibition of myelination.

In one preferred embodiment of the invention, DNA or RNA encoding prosaposin or a prosaposin receptor agonist is placed in a eukaryotic expression vector for ex vivo transfection or infection of neural cells obtained from an individual with a neurodegenerative or myelination disorder. Such transfected or infected cells are then re-introduced into the patient. Such cells include Schwann cells, oligodendrocytes, glial cells, astrocytes and dendrocytes. These transfected cells may be implanted into the appropriate neural site, including the brain, cerebrospinal fluid and peripheral nerves.

Neurons and glia can be derived from a common fetal precursor cell (McKay, Science 276:66-71, 1997). The adult nervous system also contains multipotential precursors for neurons, astrocytes and oligodendrocytes (Reynolds et al., Science 255:1707, 1992; Gritti et al., J. Neurosci. 16:1091, 1995; Johe et al., Genes Dev. 10:3129, 1996). Cultured cells of both the adult and fetal CNS that have proliferated in vitro can differentiate to show morphological and electrophysiological features characteristic of neurons: regenerative and synaptic structures (Gritti et al., supra.; Vicario-Abejon et al., Neuron 15:105, 1995; McKay et al., supra.).

In another preferred embodiment, the multipotential neural stem cells disclosed above are obtained from a mammal, preferably a human, cultured ex vivo, transfected or infected with an expression vector encoding prosaposin or a prosaposin receptor agonist, and reintroduced into the mammal. The stem cells containing the protein or peptide then differentiate into a particular neural cell type and continuously produce the peptide.

Many such eukaryotic expression vectors are known and commercially available. Standard techniques for the construction of these expression vectors are well known and can be found in references such as Sambrook et al., Molecular Cloning: A Laboratory Manual, Cold Spring Harbor Laboratory Press, Cold Spring Harbor, N.Y., 1989, or in any of the widely available laboratory manuals on recombinant DNA technology. A variety of strategies are available for ligating fragments of DNA. the choice of which depends on the nature of the termini of the DNA fragments and can be readily determined by one of ordinary skill in the art.

Preferred expression vectors include viral vectors such as retroviral vectors, adenoviral vectors and adeno-associated viral vectors. Herpesvirus vectors may also be used. These viruses do not integrate their genes into the host DNA; however, they are attracted to neurons, some of which retain the viruses and the exogenous DNA sequences contained therein in a more or less innocuous state. The use of herpesvirus vectors is therefore desirable for therapy aimed at neurological disorders. These commonly used vectors for gene therapy are discussed in detail by Miller et al. (FASEB J., 9:190-199, 1995).

The expression vector also typically contains a selectable marker, such as antibiotic resistance, to select for cells which are expressing the therapeutic protein or peptide. Although the preferred method of ex vivo cell transfection is electroporation, other methods are also contemplated including calcium phosphate precipitation, microinjection and cell fusion. Gene delivery systems are described by Felgner et al. (Hum. Gene Ther. 8:511-512, 1997) and include cationic lipid-based delivery systems (lipoplex), polycation-based delivery systems (polyplex) and a combination thereof (lipopolyplex), all of which are contemplated for use in the present invention.

Expression vector constructs containing DNA or RNA encoding prosaposin, saposin C, a neurotrophic peptide derived therefrom or a prosaposin receptor agonist, can be administered in vivo to neuronal cells by two techniques. In the first technique, gene therapy is carried out ex vivo in a procedure in which an expression cassette is transferred to cells from an individual with a neural or myelination disorder in the laboratory by standard transfection or infection methods and the modified cells are then returned to the individual. Alternatively, gene transfer can be done in vivo by transferring the expression cassette directly to cells within an individual. In both cases, the transfer process is usually facilitated by a vector that helps deliver the cassette to the intracellular site where it can function appropriately. Vector systems for gene therapy are discussed in detail by Hodgson (Exp. Opin. Ther. Patents, 5:459-468, 1995). The expression cassette typically contains an appropriate heterologous promoter for driving expression of the gene. Such promoters are well known in the art and include, for example, the SV40 and cytomegalovirus (CMV) promoters. The use of constitutive, inducible and tissue-specific promoters are all within the scope of the present invention. Other nucleotide sequence elements can be incorporated into the expression vectors to facilitate integration of DNA into chromosomes, expression of the DNA and cloning of the vector. For example, the presence of enhancers upstream of the promoter or terminators downstream of the coding region can facilitate expression of the DNA or RNA contained within the expression vector.

In one embodiment of the invention, the expression vector containing the DNA or RNA of interest is injected directly into the blood. In another embodiment, the expression vector is administered by direct intracranial injection or injection into the cerebrospinal fluid. In both cases, a pharmaceutically acceptable carrier such as phosphate buffered saline (PBS) or lactated Ringer's solution is used. The appropriately coded segments of pure DNA in a pharmaceutically acceptable carrier may also be injected (“naked DNA”) rather than an expression vector containing the DNA segment. Alternatively, the composition can be administered to peripheral neural tissue by direct local injection or by systemic administration. Various conventional modes of administration are contemplated, including intravenous, intramuscular, intradermal, subcutaneous, intracerebrospinal, intracranial, epidural, topical, intranasal, transmucosal and oral.

Transfected or infected cells expressing prosaposin or a prosaposin receptor agonist can also be encapsulated in a biocompatible polymeric membrane. Examples of some of these materials are polyacrylonitrile vinyl chloride (PAN/PVC) acrylic copolymers, hydrogels such as alginate or agarose, mixed esters, cellulose, polytetrafluoroethylene/polypropylene (Lum et al., Diabetes, 40:1511-1516, 1991; Aebischer et al., Exp. Neurol., 111 :269-275, 1991; Liu et al., Hum. Gene Ther., 4:291-301, 1993; Hill et al., Cell Transplantation, 1:168, 1992) and polyethylene glycol (PEG) conformal coating configurations (U.S. Pat. No. 5,529,914). The encapsulated cells are implanted into an animal with a neurodegenerative or myelination disorder. These permselective membranes permit entry of oxygen and other essential nutrients, but exclude antibodies and cells of the immune system, thus preventing recognition of the cells as foreign and allowing the implanted cells to continually produce the neurotrophic protein or peptide. For a review of this technique, see Lanza et al., Surgery, 121:1-9, 1997. For example, the encapsulated cells are implanted within the lumbar intrathecal space in patients with amyotrophic lateral sclerosis and in the interstitial region of the brain for treatment of Parkinson's disease. Encapsulation of genetically engineered cells and implantation into mammals has been reported by several groups (Sagot et al., Eur. J. Neurosci., 7:1313-1322, 1995; Sagen et al., J. Neurosci., 13:2415-2423, 1993; Aebischer et al., Nature Medicine, 2:696-699, 1996).

The composition can be packaged and administered in unit dosage form such as an injectable composition or local preparation in a dosage amount equivalent to the daily dosage administered to a patient or as a controlled release composition. A septum sealed vial containing a daily dose of the active ingredient in either PBS or in lyophilized form is an example of a unit dosage.

In another preferred embodiment of the invention, the expression vector containing the DNA or RNA of interest is administered locally to the neural cells in vivo by implantation of the material. For example, polylactic acid, polygalactic acid, regenerated collagen, multilamellar liposomes and many other conventional depot formulations comprise bioerodible or biodegradable materials that can be formulated with biologically active compositions. These materials, when implanted, gradually break down and release the active material to the surrounding tissue. The use of bioerodible, biodegradable and other depot formulations is expressly contemplated in the present invention. Infusion pumps, matrix entrapment systems and transdermal delivery devices are also contemplated.

The DNA or RNA constructs of the present invention may also advantageously be enclosed in micelles or liposomal vectors. Liposome encapsulation technology is well known. Liposomes may be targeted to specific tissue such as neural tissue, through the use of receptors, ligands or antibodies capable of binding the targeted tissue and facilitate fusion with the plasma membrane. The preparation of these formulations is well known in the art (Radin et al., Methods Enzymol., 98:613-618, 1983). Another method of liposome preparation involves, for example, use of the Lipofectin™ and Lipofectamine™ reagents (GIBCO BRL, Gaithersburg, Md.). The DNA or RNA encoding prosaposin or a prosaposin receptor agonist may also be conjugated to a receptor ligand such as transferrin, which will transport the gene to the cell surface and/or facilitate its entry into the cell by receptor-mediated endocytosis.

The gene therapy approach of the present invention may be used to treat disorders of both the central and peripheral nervous system. Post-polio syndrome is characterized by muscle fatigue and decreased endurance with accompanying muscle weakness and atrophy. The disease is believed to be caused, in part, by the type of spinal cord motor neuron damage similar to that which occurs in amyotrophic lateral sclerosis. Peripheral nerve injuries and peripheral neuropathies, such as those resulting from diabetes or chemotherapy, comprise the most prevalent peripheral neuropathies and may be treated using the method of the present invention. Such neuropathies include spinal cord injury, macular degeneration, amyotrophic lateral sclerosis, spinal muscular atrophy, post-polio syndrome, muscular dystrophies, peripheral neuropathies, stroke and peripheral nerve injuries. Any traumatic or ischemic injury to the central or peripheral nervous system may be treated using the method of the invention.

Cells may be treated to facilitate myelin formation or to prevent demyelination in the manner described above, both in vivo and ex vivo. In ex vivo applications, the transfected neural cells are returned to the individual and will continually express the encoded prosaposin or prosaposin receptor agonist. There are several diseases of the central nervous system that result in demyelination of nerve fibers including multiple sclerosis, acute disseminated leukoencephalitis, progressive multifocal leukoencephalitis, metachromatic leukodystrophy and adrenal leukodystrophy. An example of a demyelinating disease of the peripheral nervous system is Guillain-Barré syndrome. These diseases can be treated, and the progress of the demyelination can be slowed or halted, by administration of expression vectors encoding cDNA encoding prosaposin, saposin C, a neurotrophic peptide derived from saposin C or a prosaposin receptor agonist.

Anoxia is not the ultimate event which destroys heart tissue. This process initiates apoptosis which is promoted by proinflammatory cytokines. The present method can also be used to inhibit apoptosis which occurs during cerebral infarction, myocardial infarction and congestive heart failure. As described in U.S. Provisional Application Ser. No. 60/058,352, prosaposin and prosaposin receptor agonists can be used to inhibit this apoptosis.

In another preferred embodiment, the mammal transfected with a expression vector encoding recombinant prosaposin, saposin C, or other prosaposin receptor agonist is used as a source of these materials. Prosaposin is an integral membrane and secreted protein which is found in various body fluids including milk, cerebrospinal fluid and seminal plasma. Thus, the prosaposin, saposin C or other prosaposin receptor agonist produced in vivo will be present in these body fluids which can be used as a source of these molecules. Prosaposin is purified as described in U.S. Pat. No. 5,571,787. Prosaposin receptor agonists are purified by standard affinity chromatography methods using an antibody generated against the agonist.

1. A method of treating multiple sclerosis comprising administering to a subject suffering from multiple sclerosis an effective amount of a peptide derived from SEQ ID NO:2 located within saposin C, wherein the peptide is modified to replace Lys 2 with D-Ala, to replace Lys 8 with Ala, to delete Lys 11, and to replace Asp 15 with Tyr, and wherein said peptide is expressed in implanted neural cells.

2. The method of claim 1, wherein the peptide is SEQ ID NO: 4, wherein X is D-ala.

3. The method of claim 1, wherein the disorder is selected from the group consisting of multiple sclerosis, spinal cord injury, macular degeneration, amyotrophic lateral sclerosis, spinal muscular atrophy, post-polio syndrome, muscular dystrophies, peripheral neuropathies, stroke and peripheral nerve injuries.

4. The method of claim 3, wherein the peripheral neuropathies are diabetic neuropathies.

5. The method of claim 3, wherein the peripheral neuropathies are taxol-induced neuropathies.

6. The method of claim 1, wherein the disorder is a cerebral infarct or myocardial infarct.

7. The method of claim 1, wherein the peptide is in a form suitable for an administration route selected from the group consisting of intravenous, intracerebrospinal, intramuscular, intradermal, subcutaneous, intracranial, epidural, topical, intranasal, transmucosal and oral.
